(19) United States
(12) Patent Application Publication
(10) Pub. No.: US 2021/0235993 A1
(43) Pub. Date: Aug. 5, 2021

(54) A ROBUST MEDICAL DEVICE AND METHOD

(71) Applicant: Nir Geva, Ness Ziona (IL)

(72) Inventor: Nir Geva, Ness Ziona (IL)

(21) Appl. No.: 17/051,170

(22) PCT Filed: May 1, 2019
(86) PCT No.: PCT/IB2019/053561

§ 371 Date: Oct. 27, 2020

Related U.S. Application Data
(60) Provisional application No. 62/665,173, filed on May 1, 2018.

(51) Int. Cl. A61B 5/00

(52) U.S. Cl. CPC A61B 5/0082; A61B 5/6826; A61B 5/746; A61B 5/14542; A61B 5/6843; A61B 5/0002; A61B 2562/0247; A61B 5/742

(57) ABSTRACT

A medical device that may include (a) one or more health sensors that are configured to monitor one or more parameters of a person when a finger of the person contacts the medical device; and (b) a pressure sensor for sensing a pressure applied by the finger of the person on the medical device.

### CROSS REFERENCE

This application claims priority from U.S. provisional patent 62/665,173 filing date May 1 2018 which is incorporated herein by its entirety.

### BACKGROUND

Various medical measurements involve having a person contact a medical device with his finger, directing radiation toward the finger, detecting reflected and/or transmissive radiation from the finger, and processing the detected radiation to determine a physical parameter of the person.

The person usually applies an unknown amount of pressure against the medical device. This pressure may introduce errors in the medical measurements.

### SUMMARY

There may be provided a system, a non-transitory computer readable medium, and a method for health monitoring.

There may be provided a medical device that may include one or more health sensors that may be configured to monitor one or more parameters of a person when a finger of the person contacts the medical device; and a pressure sensor for sensing a pressure applied by the finger of the person on the medical device.

The medical device may include a processor that may be configured to ignore readings of the one or more health sensors when the pressure applied by the finger of the person on the medical device may be outside an allowable pressure range.

The allowable pressure range may exclude pressure levels that result in blocking capillaries within the finger.

The processor may be configured to complete a health measurement when the pressure applied by the finger of the person on the medical device may be within the allowable pressure range.

The medical device may include a processor that may be configured to generate an alert when the pressure applied by the finger of the person on the medical device may be outside an allowable pressure range.

The medical device may include a communication unit that may be configured to transmit the alert.

The medical device may include a communication unit that may be configured to receive the alert and output a human perceivable indication.

The pressure sensor and at least one health sensor of the one or more health sensors belong to a hybrid unit of the medical device.

The pressure sensor and at least one health sensor of the one or more health sensors may be spaced apart from each other.

The one or more health sensors may include a radiation receiver and a radiation transmitter.

The pressure sensor may be positioned between the radiation receiver and the radiation transmitter.

The pressure sensor may at least partially surround the radiation receiver.

The pressure sensor may at least partially surround the radiation receiver and the radiation transmitter.

The medical device may include a body that may include an upper part and a lower part that may be rotatably coupled to each other.

The pressure sensor may be mechanically coupled to the body.

The one or more health sensors may be mechanically coupled to the body.

The one or more health sensors and the pressure sensor may be mechanically coupled to the lower part.

The medical device may include a spring that forces the upper part to move towards the lower part.

The one or more health sensors may include a radiation transmitter and a radiation receiver.

The radiation transmitter and the pressure sensor may be mechanically coupled to the lower part and wherein the radiation receiver may be mechanically coupled to the upper part.

The radiation transmitter, the radiation receiver and the pressure sensor may be mechanically coupled to the lower part.

The pressure sensor may be located between the radiation transmitter and the radiation receiver.

The upper part and the lower part may define an opening, and wherein the radiation transmitter and the radiation receiver may be closer to the opening than the pressure sensor.

The medical device may include a man machine interface.

The man machine interface may include a light emitting diode that may be configured to emit radiation of different colors that may be indicative of different pressure levels applied by the finger.

The man machine interface may include different light emitting diodes, each light emitting diode may be configured to emit radiation of a single color, wherein different colors may be indicative of different pressure levels applied by the finger.

The man machine interface may include a display that may be partitioned to different segments, whereas different segments may be allocated to different pressure levels applied by the finger.

There may be provided a non-transitory computer readable medium that stores instructions for: measuring one or more health parameters of a person when a finger of the person contacts one or more health sensors of a medical device; sensing, by a pressure sensor of the medical device, a pressure applied by the finger of the person on the medical device; determining whether the pressure applied by the finger of the person may be within an allowable pressure range; and responding to the determining.

The responding may include stopping a health measurement when the pressure applied by the finger of the person may be within the allowable pressure range.

The responding may include generating an alert indicative of the determining.

There may be provided a method for health monitoring, the method may include measuring one or more health parameters of a person when a finger of the person contacts one or more health sensors of a medical device; sensing, by a pressure sensor of the medical device, a pressure applied by the finger of the person on the medical device; determining whether the pressure applied by the finger of the person may be within an allowable pressure range; and responding to the determining.

The determining may be executed by a processor of the medical device.

The responding may include ignoring readings of the one or more health sensors when the pressure applied by the finger of the person on the medical device may be outside an allowable pressure range.

The allowable pressure range may exclude pressure levels that result in blocking capillaries within the finger.

The method may include completing a health measurement when the pressure applied by the finger of the person on the medical device may be within the allowable pressure range.

The method may include generating an alert when the pressure applied by the finger of the person on the medical device may be outside an allowable pressure range.

The method may include transmitting the alert by a communication unit of the medical device.

The method may include receiving the alert by a device that differs from the medical device and generating a human perceivable indication.

The method may include outputting, by a communication unit of the medical device, a human perceivable indication.

The method may be executed by any medical device illustrated in the specification and/or drawings.

Any combination of any steps of any method may be provided.

### BRIEF DESCRIPTION OF THE FIGURES

The subject matter regarded as the invention is particularly pointed out and distinctly claimed in the concluding portion of the specification. The invention, however, both as to organization and method of operation, together with objects, features, and advantages thereof, may best be understood by reference to the following detailed description when read with the accompanying drawings in which:

FIG. 1 is an example of a finger and of a medical device;

FIG. 2 is an example of a finger and of a medical device;

FIG. 3 is an example of a finger and of a medical device;

FIG. 4 is an example of a finger and of a medical device;

FIG. 5 is an example of a finger and of a medical device;

FIG. 6 is an example of a finger and of a medical device;

FIG. 7 is an example of a finger and of a medical device;

FIG. 8 is an example of a finger and of a medical device;

FIG. 9 is an example of a finger and of a medical device;

FIG. 10 is an example of a finger and of a medical device;

FIG. 11 is an example of a finger and of a medical device;

FIG. 12 is an example of a hybrid unit of a health monitor;

FIG. 13 illustrates an example of a method; and

FIG. 14 illustrates an example of a method.

### DETAILED DESCRIPTION OF THE FIGURES

In the following detailed description, numerous specific details are set forth in order to provide a thorough understanding of the invention. However, it will be understood by those skilled in the art that the present invention may be practiced without these specific details. In other instances, well-known methods, procedures, and components have not been described in detail so as not to obscure the present invention.

The subject matter regarded as the invention is particularly pointed out and distinctly claimed in the concluding portion of the specification. The invention, however, both as to organization and method of operation, together with objects, features, and advantages thereof, may best be understood by reference to the following detailed description when read with the accompanying drawings.

It will be appreciated that for simplicity and clarity of illustration, elements shown in the figures have not necessarily been drawn to scale. For example, the dimensions of some of the elements may be exaggerated relative to other elements for clarity. Further, where considered appropriate, reference numerals may be repeated among the figures to indicate corresponding or analogous elements.

Because the illustrated embodiments of the present invention may for the most part, be implemented using electronic components and circuits known to those skilled in the art, details will not be explained in any greater extent than that considered necessary as illustrated above, for the understanding and appreciation of the underlying concepts of the present invention and in order not to obfuscate or distract from the teachings of the present invention.

Any reference in the specification to a method should be applied mutatis mutandis to a system capable of executing the method and to a non-transitory computer readable medium that stores instructions for executing the method.

Any reference in the specification to a system should be applied mutatis mutandis to a method that may be executed by the system and to a and to a non-transitory computer readable medium that stores instructions executable by the system.

Any reference in the specification to a non-transitory computer readable medium should be applied mutatis mutandis to a method that may be execute instructions stored in the non-transitory computer readable medium and to a system that is configured to execute the instructions stored in the non-transitory computer readable medium.

There is provided a medical device that includes one or more health sensors that monitors one or more parameters of a person when a finger of the person contacts the medical device. The medical device also includes a pressure sensor for sensing the pressure applied by the user (by his finger) on the medical device. The medical device may measure, for example, CO2 levels, glucose, oxygen, and the like.

The medical measurement may proceed while the pressure applied on the medical device is within an allowable range.

The allowable range may be predefined, may be learnt by performing different medical measurements, may be tailored to the person, and the like.

The allowable range should not include pressure levels that may result in blocking the capillaries within the finger.

An indication about the pressure applied by the finger may be generated by the medical device, by another device that may receive (directly or indirectly) an indication about the pressure.

The indication (or any instructions or request aimed to the user) may be an audio and/or visual and/or tactile indication. The other device may be a mobile phone, a smart watch, a wrist band and the like.

The indication may be generated only when the pressure exceeds the allowable range, may be generated only when the pressure exceeds or about to exceed the allowed pressure range, may be generated regardless of the pressure level, may be generated when the pressure levels are within certain ranges, and the like.

The indication may be provided in addition or instead of instructions (or requests) aimed to the person—for example “please lower the pressure”, “please moderately lower the pressure”, “please substantially lower the pressure”, “please increase the pressure”, “please moderately increase the pressure”, “please substantially increase the pressure”, “please maintain the pressure”.

The medical sensor may include one or more radiation transmitters and one or more radiation receivers. There may be any spatial relationship between the medical device and the pressure sensor. The pressure sensor may be positioned between a radiation receiver and a radiation transmitter, may at least partially surround at least one radiation transmitters, may at least partially surround at least one radiation receiver, may be positioned further away from the finger in relation to the medical device, may be mechanically coupled to the medical sensor, and the like.

The medical sensor and the pressure sensors may be included in the same system on chip or in different integrated circuits.

The medical device and/or the pressure sensor may have any shape and size.

The finger usually contacts a certain area of the medical device. The pressure sensor should be included in the certain area of be mechanically coupled to the certain area.

The pressure sensor may be of any type of pressure sensor—such as piezoelectric, includes nanometric particles, inductive, capacitance based sensor.

The pressure sensor may be a discrete component—(not integrated with the medical sensor), may be integrated with the medical sensor. May be a part of a system on chip or a SOC that also includes the medical sensor.

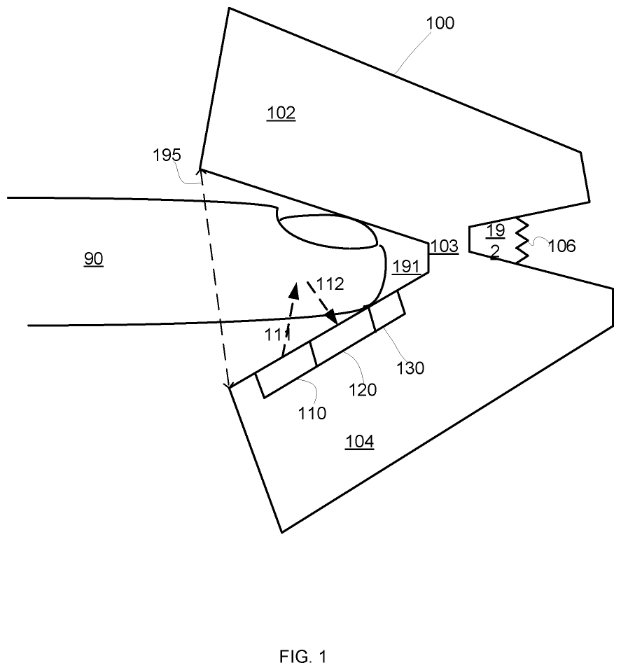

FIG. 1 illustrates a medical device 100 that includes a a clip that includes an upper part 102 and a lower part 104 that may be rotated in relation to each other, spring 106 forces the upper and lower parts 102 and 104 against finger 90, radiation transmitter 110 transmits radiation 111 towards finger 90, radiation receiver 102 receives light 112 (reflected or scattered) from the finger. Pressure sensor 130 senses the pressure applied by finger 90 on the radiation receiver.

In FIG. 1 the radiation transmitter 110, radiation receiver 102 and pressure sensor 130 form a sequence of elements. Lower part 104 is connected to or includes radiation transmitter 110, radiation receiver 102 and pressure sensor 130.

In FIG. 1 the medical device has a body that includes upper part 102, lower part 104 and intermediate part 103. The upper part 102 may be mechanically coupled to the lower part 104 via an intermediate part 103 and at least the intermediate part 103 is flexible. The upper part and the lower part may be rotatably coupled to each other in various means.

The upper part 102, lower part 104 and intermediate part 103 define (partially surround) a first inner space 191 (through which the finger may be inserted) and a second inner space 192 (in which spring 106 is located). The upper and lower parts define an opening 195 that virtually defines one of the borders of the first inner space 191.

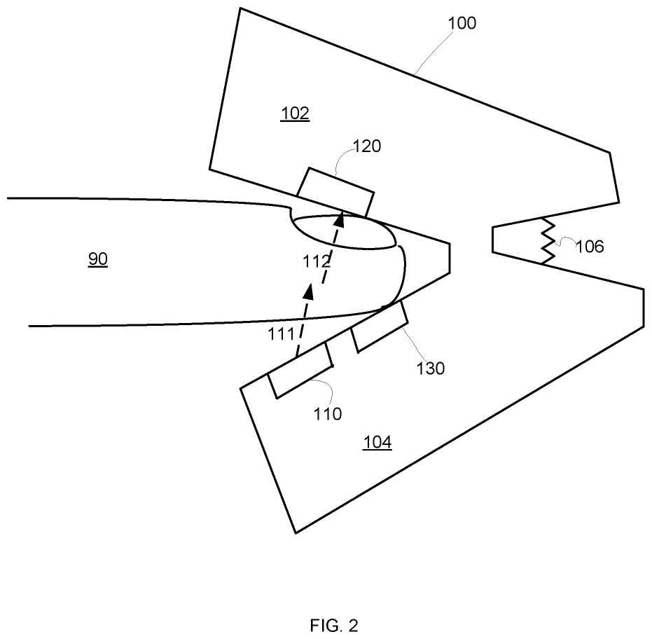

FIG. 2 illustrates finger 90 and medical device 100. The medical device of FIG. 2 differs from the medical device of FIG. 1 by having positioning the radiation receiver 102 at the upper part 102—for receiving light 112 that is transmitted through finger 90. Lower part 104 is connected to or includes radiation transmitter 110 and pressure sensor 130.

Pressure sensor 130 senses the pressure applied on the radiation transmitter.

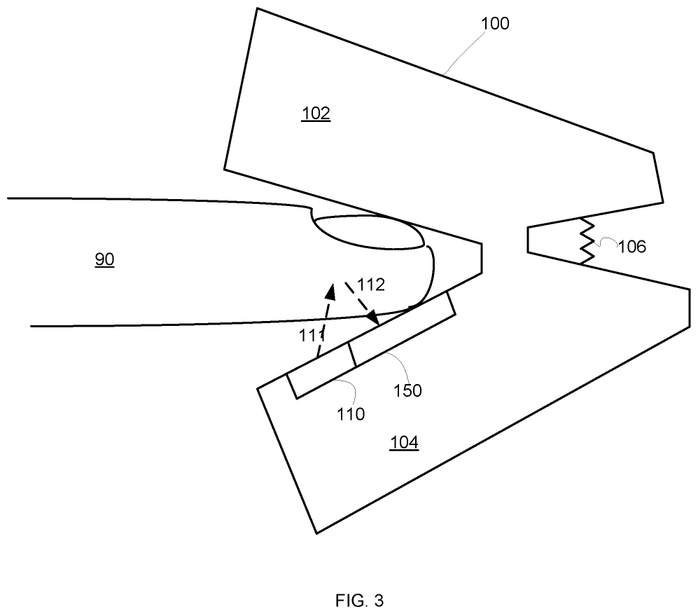

FIG. 3 illustrates finger 90 and medical device 100. The medical device of FIG. 2 differs from the medical device of FIG. 1 by having a hybrid unit 150 that includes radiation receiver 120 and pressure sensor 130.

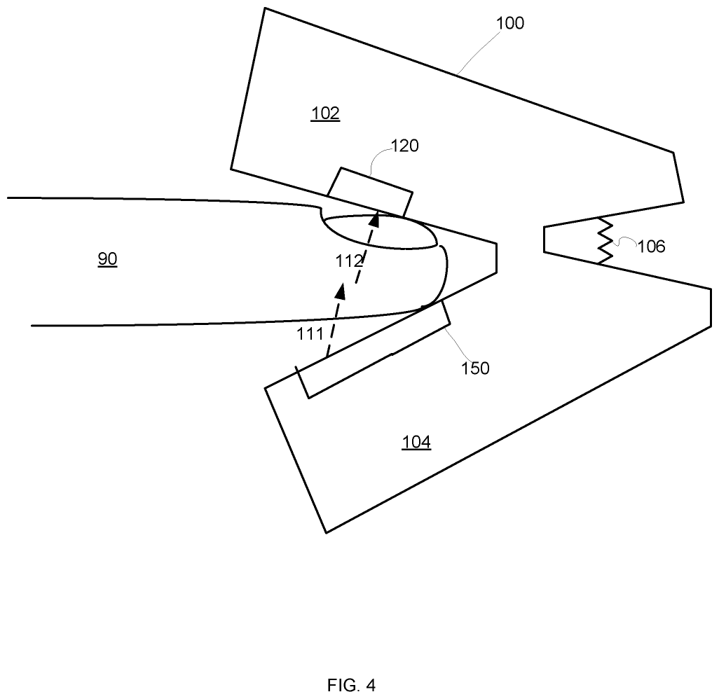

FIG. 4 illustrates finger 90 and medical device 100. The medical device of FIG. 4 differs from the medical device of FIG. 2 by having a hybrid unit 150 that includes radiation transmitter 110 and pressure sensor 130.

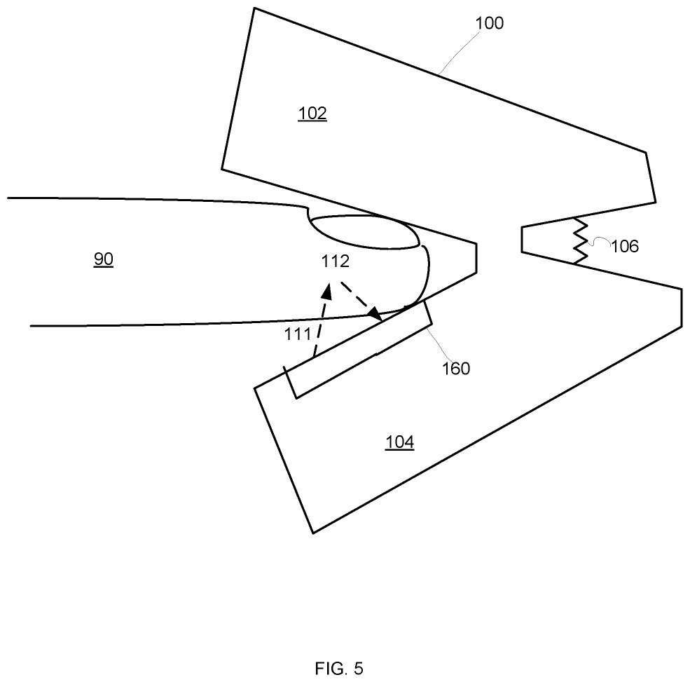

FIG. 5 illustrates finger 90 and medical device 100. The medical device of FIG. 5 differs from the medical device of FIG. 1 by having a hybrid unit 160 that includes radiation transmitter 110, radiation receiver 120 and the pressure sensor 130.

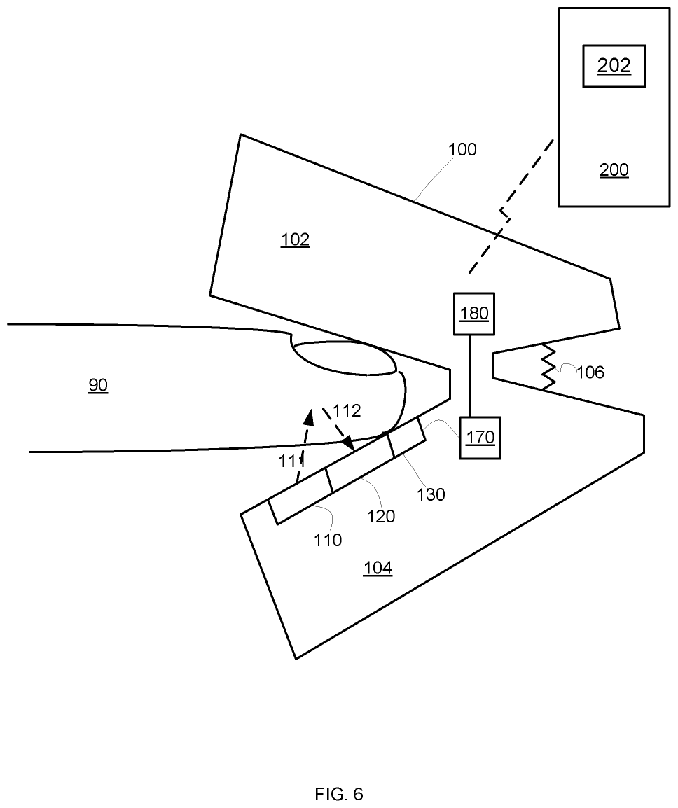

FIG. 6 illustrates finger 90 and medical device 100. The medical device of FIG. 6 differs from the medical device of FIG. 1 by having a processor 170 and a communication unit 180. Processor 170 receives pressure information from the pressure sensor 130 and determines the pressure applied by the finger. Communication unit 180 may transmit pressure information (indication, alert, pressure level) to another device 200 that has a man machine interface (MMI) 202 such as display and/or speakers and/or LEDS, and the like. Wireless communication can be used for exchanging the pressure information.

The processor may be an application-specific integrated circuit (ASIC), a field programmable gate array (FPGA), a graphic processing unit (GPU), a central processing unit (CPU), a hardware accelerator, a customized circuit, and the like.

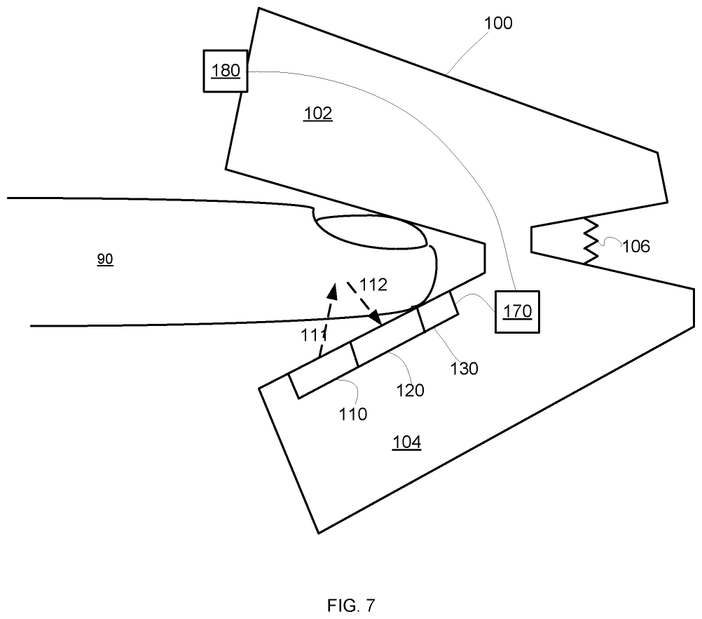

FIG. 7 illustrates finger 90 and medical device 100. The medical device of FIG. 6 differs from the medical device of FIG. 1 by having a processor 170 and an MMI 180. Processor 170 receives pressure information from the pressure sensor 130 and determines the pressure applied by the finger. MMI 180 may be instructed by processor 170 to generate audio and/or visual and/or tactile output that represents the pressure information (indication, alert, pressure level).

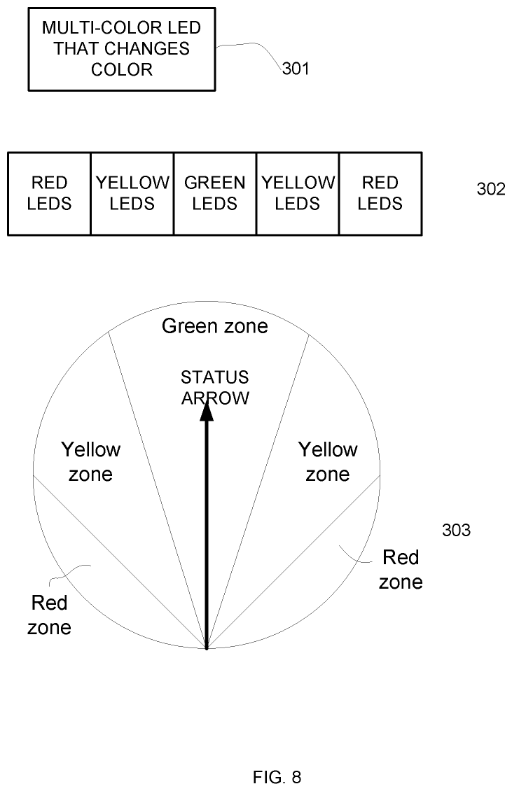

FIG. 8 illustrates various MMI elements and/or various visual indications.

The MMIs may include:
a. One or more LEDs 301 that change their color (or maybe any other parameter of illumination) to indicate the pressure level.
b. An array of LEDS of different colors that that may be activated to provide an indication about the pressure level.
c. An odometer-type display that includes a status indicative arrow (or other elements) that may be rotated to be positioned in different segments of the display.

It should be noted that using red, yellow and green colors to provide an indication of the pressure level is merely a non-limiting example of such indications.

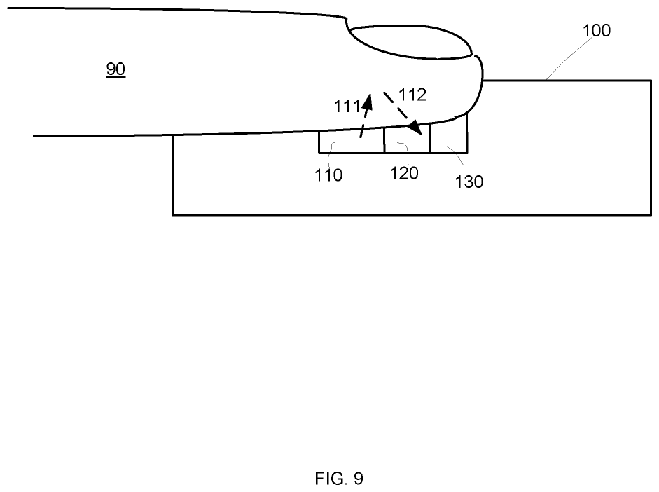

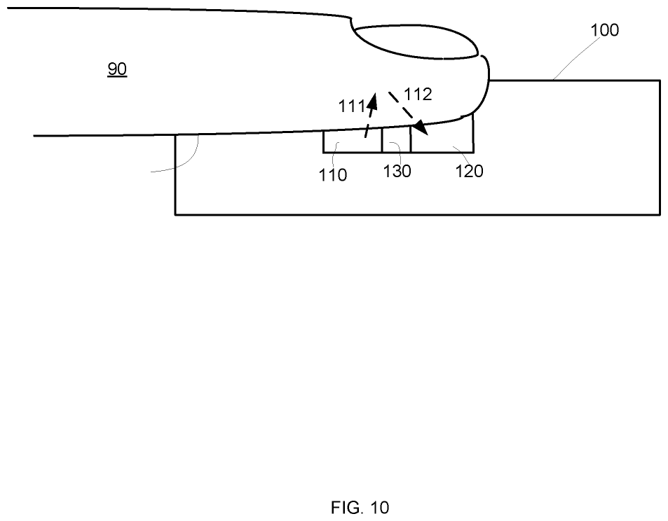

FIGS. 9 and 10 illustrates a medical device that includes a surface on which the finger is positioned. The finger is not pressed against the medical device—in contrary to the clip of FIGS. 1-7. FIGS. 9 and 10 differ from each other by the order of the pressure sensor and the radiation receiver 102.

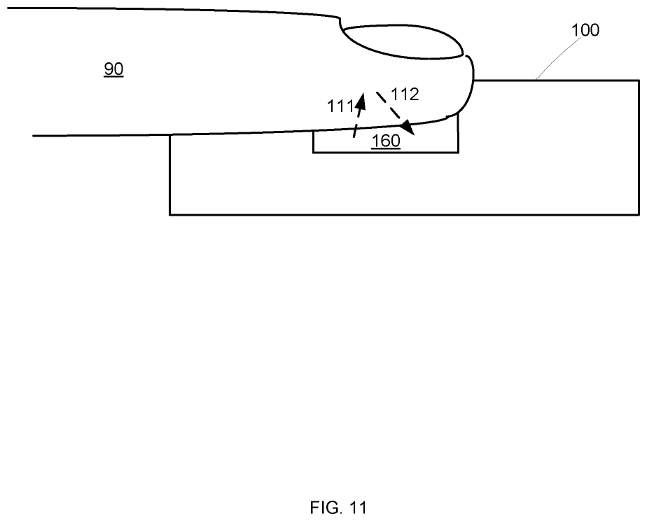

FIG. 11 illustrates finger 90 and medical device 100. The medical device of FIG. 11 differs from the medical device of FIG. 10 by having a hybrid unit 160 that includes radiation transmitter 110, radiation receiver 120 and pressure sensor 130.

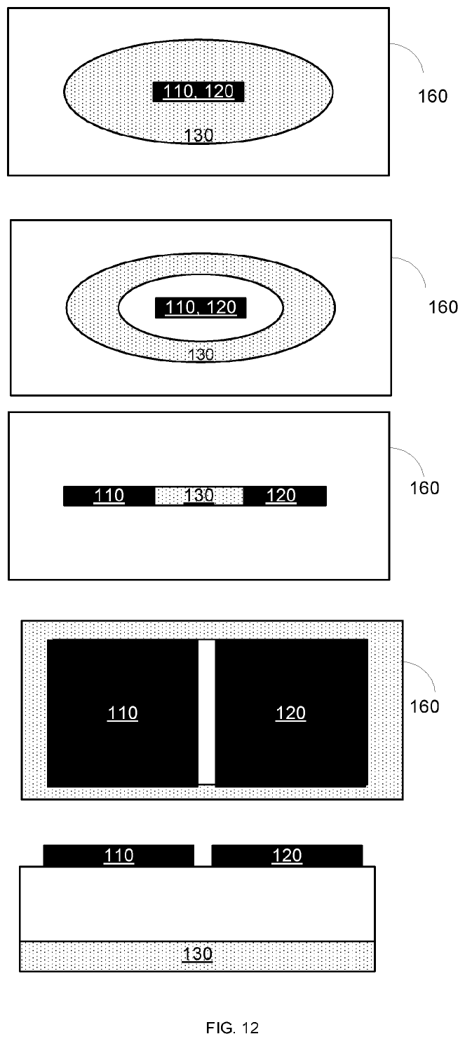

FIG. 12 illustrate various examples of the hybrid unit 160 that includes radiation transmitter 110, radiation receiver 120 and pressure sensor 130. It should be noted that hybrid unit 150 may be provided by omitting one out of radiation receiver 120 and radiation transmitter 110.

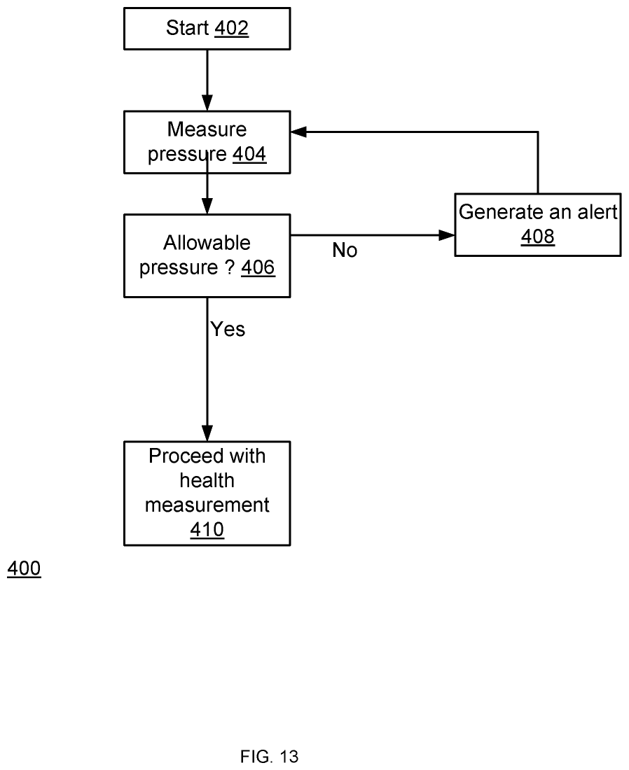

FIG. 13 illustrates method 400 that includes:
a. Start 402.
b. Performing a pressure measurement 404.
c. Checking if the pressure is within an allowable range. 406
d. If YES—proceeding with the medical measurement.
e. If NO—generating an alert and jumping to step 404.

It should be noted that the radiation transmitter may be activated before step 404 or only during step 410. The same applied to the activation of the radiation receiver 120 and/or the processing of detection signals.

Step 406 may be followed by responding to the outcome of step 406. Steps 408 and 410 are examples of such responses.

Stopping the health measurement and/or ignoring measurements obtained while the pressure is outside the allowable pressure range may save processing and/or storage resources allocated to invalid measurements.

It should be noted that the medical measurement may be executed regardless of the pressure reading—but results obtained when the pressure is outside the allowable range may be discarded or may receive a lower quality level. Alternatively—medical measurements are executed only when the pressure is in the allowed pressure range.

The number of sensors may differ from the number of sensors illustrated in any of the figures. The types of sensors differ from the types of sensors illustrated in each one of the figures.

The smartphone is merely an example of a mobile device. The mobile device may participate in processing data provided from the sensors of the health monitor, may merely transmit such the data and the like.

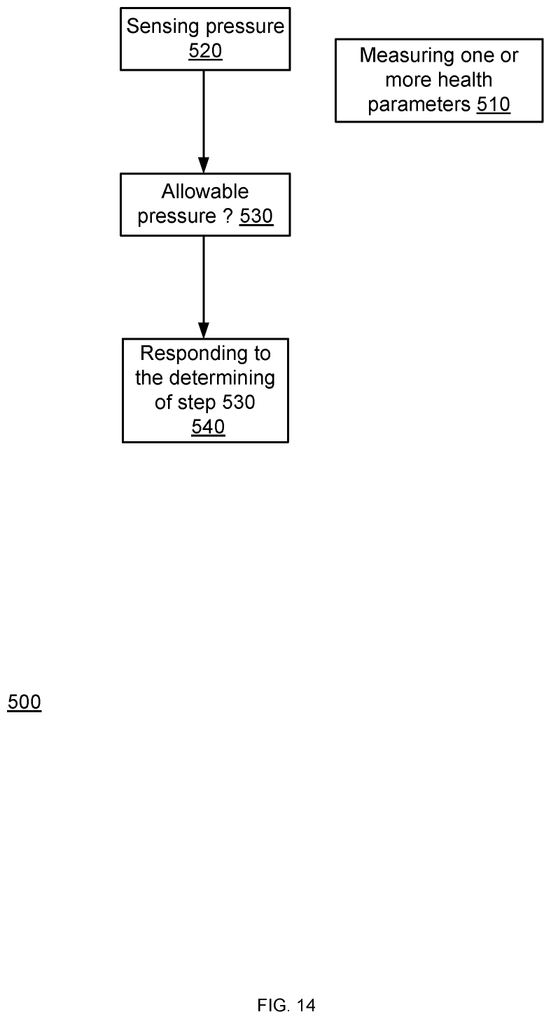

FIG. 14 illustrates method 500.

Method 500 may include the following steps:
a. Measuring one or more health parameters of a person when a finger of the person contacts one or more health sensors of a medical device 510. It should be noted that the step 510 may be conditioned by the outcome of step 530—for example starting step 510 only when the pressure applied by the finger of the person is within the allowed pressure range. Yet for another example—step 510 may be started regardless of the pressure—but may be terminated or not completed when the outcome of step 540 indicates that the pressure applied by the finger of the person is outside the allowed pressure range.
b. Sensing, by a pressure sensor of the medical device, a pressure applied by the finger of the person on the medical device 520.
c. Determining whether the pressure applied by the finger of the person is within an allowable pressure range 530.
d. Responding to the determining 540.

The determining of step 530 may be executed by a processor of the medical device—or may be executed by a processor that does not belong to the medical device.

Step 540 may include at least one of the following:
a. Ignoring readings of the one or more health sensors when the pressure applied by the finger of the person on the medical device is outside an allowable pressure range.
b. Completing a health measurement when the pressure applied by the finger of the person on the medical device is within the allowable pressure range.
c. Stopping a health measurement when the pressure applied by the finger of the person on the medical device is outside the allowable pressure range.
d. Completing the medical measurement regardless of the pressure applied by the finger and discarding results obtained when the pressure is outside the allowable range.
e. Completing the medical measurement regardless of the pressure applied by the finger and allocating a lower quality level to results obtained when the pressure is outside the allowable range. The quality level is lower in relation to the quality level allocated to results obtained when the pressure is within the allowable range.
f. Completing the medical measurements only when the pressure is in the allowed pressure range.
g. Generating an alert when the pressure applied by the finger of the person on the medical device is outside an allowable pressure range.
h. Transmitting the alert by a communication unit of the medical device.
i. Receiving the alert by a device that differs from the medical device and generating a human perceivable indication.
j. Outputting, by a communication unit of the medical device, a human perceivable indication.

There may be provided a method for operating any of the medical devices illustrated in this specification and/or drawings. The operating may include measuring oxygen saturation, and the like.

Any combination of any components and/or units illustrate din any of the figures may be provided.

Any reference to the term “comprising” or “having” should be interpreted also as referring to “consisting” of “essentially consisting of”. For example—a method that comprises certain steps can include additional steps, can be limited to the certain steps or may include additional steps that do not materially affect the basic and novel characteristics of the method—respectively.

In the foregoing specification, the invention has been described with reference to specific examples of embodiments of the invention. It will, however, be evident that various modifications and changes may be made therein without departing from the broader spirit and scope of the invention as set forth in the appended claims.

Moreover, the terms “front,” “back,” “top,” “bottom,” “over,” “under” and the like in the description and in the claims, if any, are used for descriptive purposes and not necessarily for describing permanent relative positions. It is understood that the terms so used are interchangeable under appropriate circumstances such that the embodiments of the invention described herein are, for example, capable of operation in other orientations than those illustrated or otherwise described herein.

Those skilled in the art will recognize that the boundaries between logic blocks are merely illustrative and that alternative embodiments may merge logic blocks or circuit elements or impose an alternate decomposition of functionality upon various logic blocks or circuit elements. Thus, it is to be understood that the architectures depicted herein are merely exemplary, and that in fact many other architectures may be implemented which achieve the same functionality.

Any arrangement of components to achieve the same functionality is effectively “associated” such that the desired functionality is achieved. Hence, any two components herein combined to achieve a particular functionality may be seen as “associated with” each other such that the desired functionality is achieved, irrespective of architectures or intermedical components. Likewise, any two components so associated can also be viewed as being “operably connected,” or “operably coupled,” to each other to achieve the desired functionality.

Furthermore, those skilled in the art will recognize that boundaries between the above described operations merely illustrative. The multiple operations may be combined into a single operation, a single operation may be distributed in additional operations and operations may be executed at least partially overlapping in time. Moreover, alternative embodiments may include multiple instances of a particular operation, and the order of operations may be altered in various other embodiments.

Also for example, in one embodiment, the illustrated examples may be implemented as circuitry located on a single integrated circuit or within a same device. Alternatively, the examples may be implemented as any number of separate integrated circuits or separate devices interconnected with each other in a suitable manner.

Also for example, the examples, or portions thereof, may implemented as soft or code representations of physical circuitry or of logical representations convertible into physical circuitry, such as in a hardware description language of any appropriate type.

Also, the invention is not limited to physical devices or units implemented in non-programmable hardware but can also be applied in programmable devices or units able to perform the desired device functions by operating in accordance with suitable program code, such as mainframes, minicomputers, servers, workstations, personal computers, notepads, personal digital assistants, electronic games, automotive and other embedded systems, cell phones and various other wireless devices, commonly denoted in this application as ‘computer systems’.

However, other modifications, variations and alternatives are also possible. The specifications and drawings are, accordingly, to be regarded in an illustrative rather than in a restrictive sense.

In the claims, any reference signs placed between parentheses shall not be construed as limiting the claim. The word ‘comprising’ does not exclude the presence of other elements or steps then those listed in a claim. Furthermore, the terms “a” or “an,” as used herein, are defined as one or more than one. Also, the use of introductory phrases such as “at least one” and “one or more” in the claims should not be construed to imply that the introduction of another claim element by the indefinite articles “a” or “an” limits any particular claim containing such introduced claim element to inventions containing only one such element, even when the same claim includes the introductory phrases “one or more” or “at least one” and indefinite articles such as “a” or “an.” The same holds true for the use of definite articles. Unless stated otherwise, terms such as “first” and “second” are used to arbitrarily distinguish between the elements such terms describe. Thus, these terms are not necessarily intended to indicate temporal or other prioritization of such elements. The mere fact that certain measures are recited in mutually different claims does not indicate that a combination of these measures cannot be used to advantage.

While certain features of the invention have been illustrated and described herein, many modifications, substitutions, changes, and equivalents will now occur to those of ordinary skill in the art. It is, therefore, to be understood that the appended claims are intended to cover all such modifications and changes as fall within the true spirit of the invention.

We claim:

1. A medical device, comprising one or more health sensors that are configured to monitor one or more parameters of a person when a finger of the person contacts the medical device; and a pressure sensor for sensing a pressure applied by the finger of the person on the medical device.

2. The medical device according to claim 1 further comprises a processor that is configured to ignore readings of the one or more health sensors when the pressure applied by the finger of the person on the medical device is outside an allowable pressure range.

3. The medical device according to claim 2 wherein the allowable pressure range excludes pressure levels that result in blocking capillaries within the finger.

4. The medical device according to claim 2 wherein the processor is configured to complete a health measurement when the pressure applied by the finger of the person on the medical device is within the allowable pressure range.

5. The medical device according to claim 1 comprising a processor that is configured to generate an alert when the pressure applied by the finger of the person on the medical device is outside an allowable pressure range.

6. The medical device according to claim 5 comprising a communication unit that is configured to transmit the alert.

7. The medical device according to claim 5 comprising a communication unit that is configured to receive the alert and output a human perceivable indication.

8. The medical device according to claim 1 wherein the pressure sensor and at least one health sensor of the one or more health sensors belong to a hybrid unit of the medical device.

9. The medical device according to claim 1 wherein the pressure sensor and at least one health sensor of the one or more health sensors are spaced apart from each other.

10. The medical device according to claim 1 wherein the one or more health sensors comprise a radiation receiver and a radiation transmitter.

11. The medical device according to claim 10 wherein the pressure sensor is positioned between the radiation receiver and the radiation transmitter.

12. The medical device according to claim 10 wherein the pressure sensor at least partially surround the radiation receiver.

13. The medical device according to claim 10 wherein the pressure sensor at least partially surround the radiation receiver and the radiation transmitter.

14. The medical device according to claim 1 comprising a body that comprises an upper part and a lower part that are rotatably coupled to each other.

15. The medical device according to claim 14 wherein the pressure sensor is mechanically coupled to the body.

16. The medical device according to claim 14 wherein the one or more health sensors are mechanically coupled to the body.

17. The medical device according to claim 14 wherein the one or more health sensors and the pressure sensor are mechanically coupled to the lower part.

18. The medical device according to claim 14 comprising a spring that forces the upper part to move towards the lower part.

19. The medical device according to claim 14 wherein the one or more health sensors comprise a radiation transmitter and a radiation receiver.

20. The medical device according to claim 19 wherein the radiation transmitter and the pressure sensor are mechanically coupled to the lower part and wherein the radiation receiver is mechanically coupled to the upper part.

21. The medical device according to claim 19 wherein the radiation transmitter, the radiation receiver and the pressure sensor are mechanically coupled to the lower part.

22. The medical device according to claim 21 wherein the pressure sensor is located between the radiation transmitter and the radiation receiver.

23. The medical device according to claim 21 wherein the upper part and the lower part define an opening, and wherein the radiation transmitter and the radiation receiver are closer to the opening than the pressure sensor.

24. The medical device according to claim 1 comprising a man machine interface.

25. The medical device according to claim 24 wherein the man machine interface comprises a light emitting diode that is configured to emit radiation of different colors that are indicative of different pressure levels applied by the finger.

26. The medical device according to claim 24 wherein the man machine interface comprises different light emitting diodes, each light emitting diode is configured to emit radiation of a single color, wherein different colors are indicative of different pressure levels applied by the finger.

27. The medical device according to claim 24 wherein the man machine interface comprises a display that is partitioned to different segments, whereas different segments are allocated to different pressure levels applied by the finger.

28. A non-transitory computer readable medium that stores instructions for:
measuring one or more health parameters of a person when a finger of the person contacts one or more health sensors of a medical device;
sensing, by a pressure sensor of the medical device, a pressure applied by the finger of the person on the medical device;
determining whether the pressure applied by the finger of the person is within an allowable pressure range; and
responding to the determining.

29. The non-transitory computer readable medium according to claim 28 wherein the responding comprises stopping a health measurement when the pressure applied by the finger of the person is within the allowable pressure range.

30. The non-transitory computer readable medium according to claim 28 wherein the responding comprises generating an alert indicative of the determining.

31. A method for health monitoring, the method comprises:
measuring one or more health parameters of a person when a finger of the person contacts one or more health sensors of a medical device;
sensing, by a pressure sensor of the medical device, a pressure applied by the finger of the person on the medical device;
determining whether the pressure applied by the finger of the person is within an allowable pressure range; and
responding to the determining.

32. The method according to claim 31 wherein the determining is executed by a processor of the medical device.

33. The method according to claim 31 wherein the responding comprises ignoring readings of the one or more health sensors when the pressure applied by the finger of the person on the medical device is outside an allowable pressure range.

34. The method according to claim 32 wherein the allowable pressure range excludes pressure levels that result in blocking capillaries within the finger.

35. The method according to claim 32 comprising completing a health measurement when the pressure applied by the finger of the person on the medical device is within the allowable pressure range.

36. The method according to claim 31 comprising generating an alert when the pressure applied by the finger of the person on the medical device is outside an allowable pressure range.

37. The method according to claim 35 comprising transmitting the alert by a communication unit of the medical device.

38. The method according to claim 37 comprising receiving the alert by a device that differs from the medical device and generating a human perceivable indication.

39. The method according to claim 35 outputting, by a communication unit of the medical device, a human perceivable indication.
